(19) United States
(12) Patent Application Publication
(10) Pub. No.: US 2020/0289180 A1
(43) Pub. Date: Sep. 17, 2020

(54) Assembly Formed By A Bone Anchoring Base Belonging to a Prosthetic Joint and by at Least One Screw Fastening This Base to a Bone

(71) Applicant: FX Shoulder USA Inc., Dallas, TX (US)

(72) Inventors: Baptiste Martin, Dallas, TX (US); David Louis Glaser, Villanova, PA (US); Frederick Suh Song, Princeton, NJ (US); Alfred Cook, Mount Dora, FL (US); Mark D'Onofrio, Columbus, OH (US)

(21) Appl. No.: 16/817,144

(22) Filed: Mar. 12, 2020

(30) Foreign Application Priority Data
Mar. 12, 2019 (FR) FR1902535

(51) Int. Cl. A61B 17/86; A61B 17/68

(52) U.S. Cl. CPC A61B 17/864; A61B 17/685

(57) ABSTRACT

An assembly has a bone anchoring base and a screw. The bone anchoring base has at least one protrusion configured to be inserted in a hole arranged in the cancellous portion of a bone. The screw has a thread suitable for engaging with the cancellous portion of the bone. The protrusion is inwardly hollow and comprises an orifice at its distal free end, the screw being configured to be engaged through the protrusion and screwed in the cancellous portion of the bone through the orifice. The orifice is tapped at the pitch of the thread of the screw and the thickness of a distal wall is such that it is possible to arrange more than half of a turn of the threading therein, suitable for receiving the thread of the screw, such that, during the placement of the screw, the thread of this screw engages with said distal wall.

### CROSS-REFERENCE TO RELATED APPLICATIONS

This application claims priority under 35 U.S.C. § 119 to French patent application FR 1902535, filed Mar. 12, 2019, the disclosure of which is incorporated herein by reference in its entirety.

### TECHNICAL FIELD

The present invention relates generally to a joint assembly, and, more particularly, to an assembly formed by a bone anchoring base belonging to a prosthetic joint and by at least one screw fastening this base to a bone.

### BACKGROUND

A prosthetic joint frequently comprises two prosthetic parts fastened to the respective bones of the treated joint, at least one of these parts comprising a bone anchoring base and an articular part fastened to this base. The bone anchoring base frequently comprises at least one protrusion intended to be inserted into a hole arranged in the cancellous bone of the corresponding bone, in particular in the form of an anchoring stud. The base in question may in particular be a prosthetic shoulder glenoid or a prosthetic knee tibial plateau. The articular part placed on this bone anchoring base is intended to form an articular surface conjugated with the articular surface formed by the other part of the prosthesis; for a shoulder prosthesis, it involves a sliding plate or a glenosphere, and for a knee prosthesis, it involves the sliding plate reforming a tibial plateau.

The insertion of the anchoring stud in the cancellous bone makes it possible to guarantee a certain immobilization of the base relative to the bone, but it is not sufficient to withstand all of the repeated forces exerted on the prosthesis part due to movements by the patient; it is therefore necessary to guarantee this immobilization using one or several anchoring screws passing through the base.

It is in particular known to provide that at least one protrusion formed by the base for anchoring thereof to the bone is inwardly hollow and that the protrusion is pierced by an orifice at its distal free end, said screw being intended to be engaged through the protrusion, then to be screwed into cancellous bone through said orifice until the head of the screw comes into contact with the distal wall of the protrusion defining the free end of this protrusion.

Such a prosthesis has the drawback of a risk of the anchoring screw, during the placement thereof, not being perfectly coaxial with the protrusion, which, during tightening of the screw, creates tensions between the base and the bone that are not very favorable to the proper anchoring of the prosthesis to the bone. Furthermore, in case of poor quality cancellous bone, the placement of the screw may lead to damage of this bone by the thread of the screw during the placement of this screw, particularly if the screwing process is continued while the head of the screw has come into contact with the distal wall of the protrusion defining the free end of this protrusion; this results in insufficient anchoring of the screw in the bone, and therefore insufficient anchoring of the base to the bone.

The disclosed assembly aims to resolve these and other drawbacks of the prior art.

### SUMMARY OF THE INVENTION

In the assembly in question, the screw has a thread suitable for engaging with the cancellous bone in a manner known in itself, and the bone anchoring base forms at least one protrusion intended to be inserted in a hole arranged in the cancellous bone of the corresponding bone, this protrusion being inwardly hollow and being pierced by an orifice at its distal free end, the screw being intended to be engaged through the protrusion, then to be screwed into the cancellous bone through said orifice until the head of the screw comes into contact with the distal wall of the protrusion defined by the free end of this protrusion.

According to at least some embodiments, said orifice is tapped at the pitch of the thread of the screw and the thickness of said distal wall is such that it is possible to arrange more than half of a turn of the threading therein suitable for receiving the thread of the screw, such that, during the placement of the screw, the thread of this screw engages by screwing with said distal wall.

The assembly according to the at least some embodiments thus makes it possible for the screw, during its placement, to engage by screwing with the part of said distal wall defining said orifice, which makes it possible not only to guide the screw perfectly in a manner coaxial to said anchoring protrusion, but also to advance the screw in the bone while this screw is being screwed in and to block the rotation of the screw when the head of the screw encounters said distal pierced wall at the end of screwing.

The fact that the screw is placed perfectly coaxially to the anchoring protrusion during the placement of the screw eliminates the risk of tensions being exerted between the bone and the anchoring base; the fact that an advance of the screw occurs by the screwing engagement during screwing, as well as the fact that the rotation of the screw is blocked at the end of screwing, effectively prevents the risk of damage to the portion of the cancellous bone into which the screw is intended to be inserted.

As a result, all of the aforementioned drawbacks of the system according to the prior art are thus resolved.

Preferably, the thickness of said distal wall is such that it is possible to arrange at least one complete threading turn therein suitable for receiving the thread of the screw.

A perfect screwing engagement is thus produced between the screw and the bone anchoring base.

Preferably, said bone anchoring base is a prosthetic shoulder glenoid base, said protrusion being an anchoring stud arranged coaxially to a plate formed by this base.

The assembly according to at least some embodiments thus allows ideal placement of such a glenoid base on a scapula, which is important for the proper operation of the prosthetic shoulder.

### BRIEF DESCRIPTION OF THE DRAWINGS

The foregoing and other aspects of the present invention are best understood from the following detailed description when read in connection with the accompanying drawings. For the purpose of illustrating the invention, there are shown in the drawings embodiments that are presently preferred, it being understood, however, that the invention is not limited to the specific instrumentalities disclosed. Included in the drawings are the following Figures:

FIG. 1 is a perspective view of a prosthetic shoulder glenoid base and a screw intended to be screwed into the cancellous bone inserted through an anchoring protrusion comprised by the glenoid base;

FIG. 2 is an axial view of the glenoid base on the proximal side;

FIG. 3 is a view of the glenoid base from the side, in sectional view along the axis of this base, a detail being circled at the distal end of said anchoring protrusion;

FIG. 4 is an enlarged view of the detail circled in FIG. 3;

FIG. 5 is a view of the glenoid base similar to FIG. 3, during the placement of the screw; and

FIG. 6 is a view of the base and the screw similar to FIG. 5 once the screw is completely screwed in.

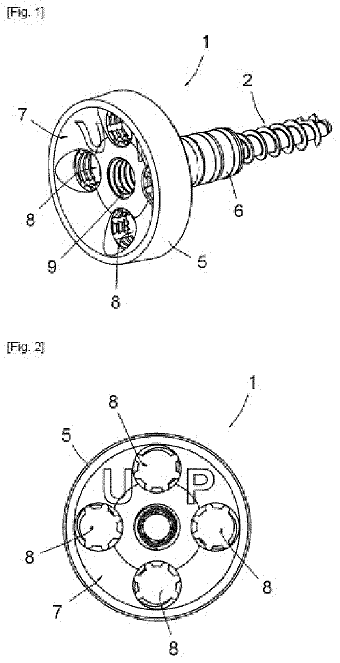

### DETAILED DESCRIPTION

The figures show a glenoid base 1 of a prosthetic shoulder and a screw 2 intended to be screwed into the cancellous bone of a scapula in order to guarantee perfect anchoring of the base 1 to said scapula.

The base 1 comprises a plate 5 intended to receive an articular part (not shown) and a protrusion 6 of a piece with this plate, intended to be inserted tightly into a hole previously arranged in the cancellous bone of the scapula.

The plate 5 forms a proximal receiving housing 7, by force or with clipping, for said articular part, which may in particular be made from high-density polyethylene to guarantee sliding with the conjugated articular head of the part affixed to the end of the humerus.

In the illustrated example, the plate 5 comprises four holes 8 for receiving bone anchoring screws arranged through it, surrounding the protrusion 6. These holes 8 may be of the type described in French patent application No. FR 1,154,791 (U.S. Pat. No. 2,975,888), which is herein incorporated by reference in its entirety, having radial notches that guarantee a specific orientation of anchoring screws during the placement of these screws.

Furthermore, the plate 5 is inwardly etched with an inscription making it possible to identify the portion of this plate having to be placed at an angle against the upper position of the scapula, namely “UP” in the illustrated example.

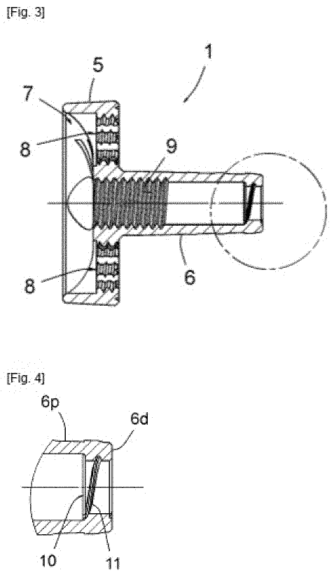

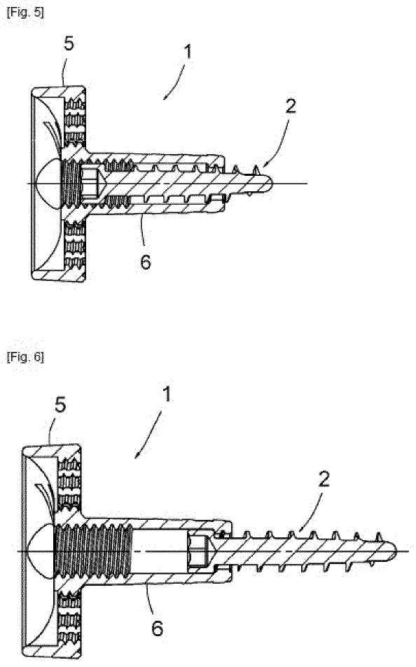

The protrusion 6 is secured to the central portion of the plate 5. It is inwardly hollow and emerges at the center of the plate 5. It has outer reliefs allowing a certain axial retention relative to the cancellous bone of the scapula into which it is inserted. Inwardly, as particularly visible in FIG. 3, it has a proximal mechanical threading 9 allowing its connection to the corresponding threaded end of the shaft of a tool (not shown) for grasping, manipulating and impacting the base 1, well known in itself. At its distal end, as particularly visible in FIG. 4, the protrusion 6 has a distal wall 6d with a thickness greater than the peripheral wall 6p of this protrusion, this distal wall 6d being pierced by an orifice 10. This orifice 10 allows the passage of the screw 2, as shown by FIGS. 5 and 6.

The orifice 10 is tapped at the pitch of the thread of the screw 2, and FIG. 4 shows that the thickness of the wall 6d is such that it is possible to arrange a complete threading turn 11 therein suitable for receiving the thread of the screw 2. Thus, during the placement of the screw 2, the thread of this screw engages by screwing with the wall 6d.

The screw 2 in turn is of the traditional type, having an outwardly smooth a cylindrical head with a diameter that is slightly smaller than the inner diameter of the protrusion 6.

As is understood, the tapping of the orifice 10 makes it possible for the screw 2, during placement thereof, to engage by screwing with the part of the wall 6d defining the orifice 10, which makes it possible not only to guide the screw 2 perfectly in a manner coaxial to the protrusion 6, but also to advance the screw 2 in the cancellous bone while this screw is being screwed in and to block the rotation of the screw 2 when the head of the screw encounters said wall 6d at the end of screwing.

The disclosed embodiments thus provide an assembly with the decisive advantage of resolving the aforementioned drawbacks of the homologous assemblies according to the prior art.

The disclosed embodiments have been described in reference to a glenoid base 1; it must, however, be understood that it is applicable to other types of bone anchoring bases belonging to a prosthetic joint, in particular to a tibial base of a prosthetic knee.

The elements of the figures are not exclusive. Other embodiments may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention.

1. An assembly comprising:
a bone anchoring base of a prosthetic joint comprising at least one protrusion configured to be inserted in a hole arranged in the cancellous portion of a bone; and
at least one screw configured to fasten the base to the bone, wherein the screw has a thread configured to engage in the cancellous portion of the bone,
wherein the protrusion is inwardly hollow and comprises an orifice at its distal free end,
wherein the screw is configured to pass through the hollow protrusion, and be screwed in the cancellous portion of the bone through said orifice until a head of the screw comes into contact with a distal wall of the protrusion defined by a free end of the protrusion,
wherein said orifice is tapped at the pitch of the thread of the screw and
wherein the thickness of said distal wall is such that more than half of a turn of the threading suitable for receiving the thread of the screw is formed therein such that, during the placement of the screw, the thread of the screw engages with said distal wall.

2. The assembly according to claim 1, wherein the thickness of the distal wall is such that at least one complete threading turn suitable for receiving the thread of the screw is formed therein.

3. The assembly according to claim 1, wherein the bone anchoring base comprises a plate.

4. The assembly according to claim 3, wherein the at least one protrusion extends from the plate.

5. The assembly according to claim 3, wherein said bone anchoring base is a prosthetic shoulder glenoid base

6. The assembly according to claim 5, wherein said protrusion is an anchoring stud arranged coaxially to the plate formed by the bone anchoring base.
